(19) United States
(12) Patent Application Publication
(10) Pub. No.: US 2010/0256445 A1
(43) Pub. Date: Oct. 7, 2010

(54) Endoscopic Working Channel and Method of Making Same

(75) Inventor: Craig Fitzpatrick, Phoenix, AZ (US)

Correspondence Address: ETHERTON LAW GROUP, LLC, 2010 E. University Drive, Suite 25, Tempe, AZ, 85281, US

(73) Assignee: INTERNATIONAL POLYMER ENGINEERING, INC., Tempe, AZ (US)

(21) Appl. No.: 12/819,075

(22) Filed: Jun. 18, 2010

Related U.S. Application Data
(63) Continuation-in-part of application No. 11/636,270, filed on Dec. 7, 2006.

(51) Int. Cl. A61B 1/00; B32B 37/02

(52) U.S. Cl. 600/101; 156/294

(57) ABSTRACT

A working channel for an endoscope or similar device having concentric porous and nonporous polymer tubes that are adhered or bonded together. A support wire may be wrapped around a nonporous inner tube and provides increased flexibility and resistance to compression and kinking. A porous outer tube fits over the support wire and inner tube and bonds to the inner tube, encapsulating the support wire. The outer tube may have a smooth or corrugated exterior, depending on the intended use of the working channel. Methods for manufacturing the working channel are also disclosed.

### CROSS-REFERENCE TO RELATED APPLICATIONS

This application is a continuation-in-part of and claims the benefit of co-pending U.S. patent Ser. No. 11/636,270 filed Dec. 7, 2006.

### FIELD OF INVENTION

This invention relates to endoscopic devices. This invention relates particularly to an apparatus for providing a working channel within an endoscope.

### BACKGROUND

The field of endoscopy includes the use of tubular structures inserted intraluminally into a mammalian body cavity for visualizing, treating, and taking a biopsy of tissue regions within the mammalian body. Most endoscopes currently include at least one of a plurality of working channels which extend along the length of the endoscope to provide access to body tissue within the mammalian body cavity. These working channels typically include a rigid non-bendable section and a flexible bendable section. The working channels allow for air insufflation, water flow, suction, and biopsies. Conventional endoscopes utilize a wide variety of materials for the working channels, but all conventional endoscopes require the endoscopic working channel to be an integral part of the endoscope.

Because endoscopes are subjected to repeated use and are required to follow tortuous pathways within the body, a frequent cause of failure of the endoscope working channel is the bending, kinking or fracture of a section of the working channel. This renders the endoscope useless until it is repaired. Unfortunately, repair of the endoscopic working channel requires disassembly of the endoscope and replacement of the endoscope working channel.

Another problem confronting reusability of working channels is the potential for residual contaminants on the working channel after a procedure. Most working channels prevent the permeation of contaminants by making the channel out of nonporous materials only. Unfortunately, such materials are susceptible to kinking, exacerbating this cause of failure. A working channel that is impermeable to contaminants and resistant to kinking and ovaling is needed.

The endoscopic working channel of U.S. Pat. No. 5,885,209 is designed to be retrofitted as a replacement bendable section of the working channel of an endoscope. The structure of the endoscopic working channel of this patent, however, is relatively complex and is relatively expensive to manufacture because it requires mixing and maintaining very specific liquid elastomer coatings that have significant materials and labor costs. It is desirable to provide an improved endoscopic working channel that is simpler and less expensive to manufacture.

Attempted solutions to the kinking problem have corrugated the working channel in order to provide flexibility and support while the working channel is bent. However, in certain applications this corrugation may be unwanted by the user due to an increased chance of catching a part of the corrugated surface on a body part or another surgery tool or harboring contaminants. It is desirable to provide a working channel that has a smooth exterior surface that prevent such dangers.

Therefore, it is an object of this invention to provide a working channel for an endoscope that may be used to replace a broken working channel. It is a further object that the working channel be easy to manufacture and cost-effective as a replacement channel. It is a further object that the working channel protect its interior from contaminants while being highly resistant to kinking and other unwanted compression. Another object of the invention is to provide a method of manufacture whereby a working channel may be produced with either a corrugated or smooth external surface.

### SUMMARY OF THE INVENTION

An improved working channel for an endoscope or similar device has concentric polymer tubes that are adhered or bonded together. The inner tube is a nonporous material that prevents permeation of contaminants, and the outer tube is a porous material that gives the working channel flexibility while supporting the inner tube against kinking and ovaling. The outer tube may have a smooth or corrugated external surface. A support wire may be encapsulated between the inner tube and outer tube to reinforce the channel. Methods for manufacturing the working channel include using a compression die to bond the inner tube and outer tube, and wrapping a compression wrap around the outer tube before sintering the tubes.

### BRIEF DESCRIPTION OF THE DRAWINGS

FIG. 1 is a perspective partial cutaway view of a working channel with a smooth external surface and no support wire.

FIG. 2 is a perspective partial cutaway view of a working channel with a corrugated external surface and a support wire.

FIG. 3A is a cross-sectional view of an embodiment of a working channel with no support wire, showing the working channel of FIG. 1 after the compression wrap is removed.

FIG. 3B is a cross-sectional view of an embodiment of a working channel with a support wire, showing a smooth external surface.

FIG. 3C is a cross-sectional view of another embodiment of a working channel, showing an external surface that is corrugated using a closely-spaced round wire.

FIG. 3D is a cross-sectional view of another embodiment of a working channel, showing an external surface that is corrugated using a closely-spaced flat wire.

FIG. 3E is a cross-sectional view of another embodiment of a working channel, showing an external surface that is corrugated using a widely-spaced flat wire.

FIG. 4 is a process flow diagram illustrating a method used to manufacture the working channel.

FIGS. 5A-B are process flow diagrams illustrating methods for bonding the inner and outer tubes.

FIG. 6 is a process flow diagram illustrating the preferred method used to manufacture the working channel.

### DETAILED DESCRIPTION OF THE INVENTION

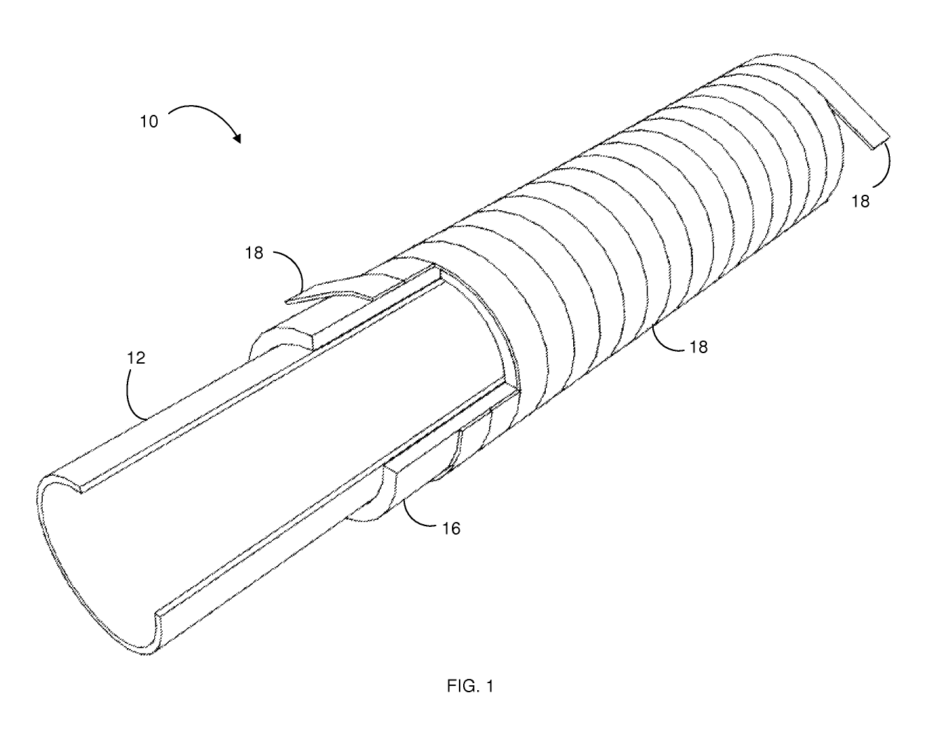

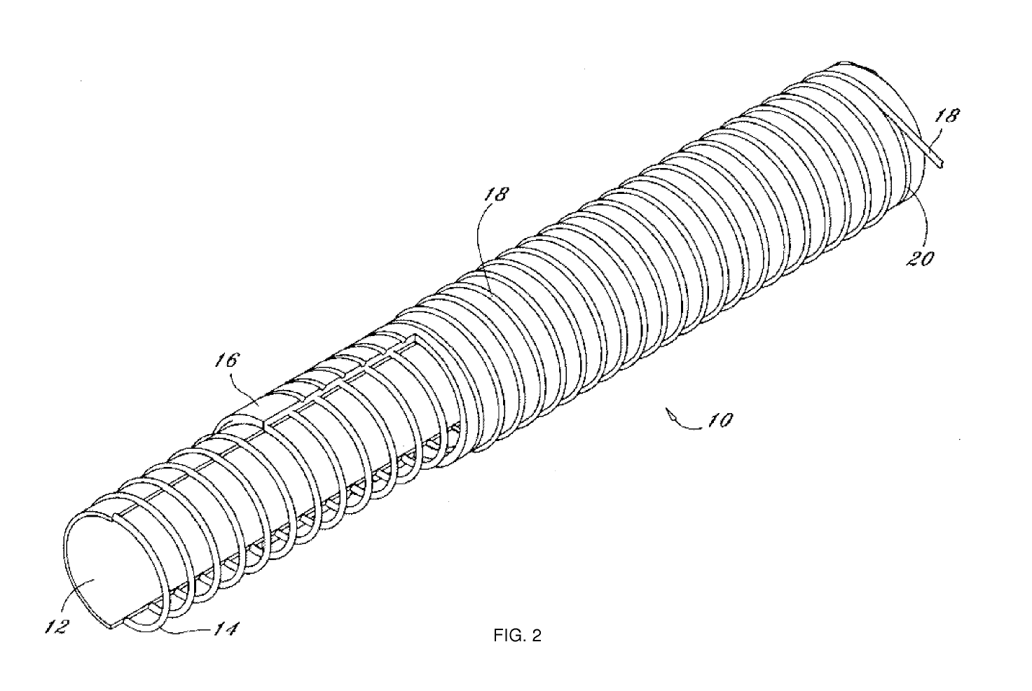

Reference now should be made to the drawings, in which the same reference numbers are used throughout the different figures to designate the same or similar components. FIGS. 1 and 2 illustrate two embodiments of the present invention, designated generally as 10, which is a working channel having an inner tube 12 bonded to an outer tube 16. The inner tube 12 is fabricated from a nonporous sintered or extruded polymer, preferably sintered, non-expanded polytetrafluoroethylene (“PTFE”). Typically, the density of the inner tube 12 is in the range of 1.8 to 2.2 g/cc. The wall thickness of the inner tube 12 may be between 0.001 inches and 0.007 inches when the working channel 10 is to be used in an endoscope. In the preferred embodiment, the wall thickness is between 0.003 and 0.004 inches. Other densities and thicknesses may be favorable for other applications, such as cable and instrument covers and chemical-resistant delivery tubes.

The outer tube 16 is a tube of porous polymer, preferably expanded PTFE (“ePTFE”), that is made by extrusion or other means, and is preferably solidified by sintering. The outer tube 16 has an internal diameter that encompasses the inner tube 12 and is in a close relationship to the external diameter of the inner tube 12. The wall thickness of the outer tube 16 is dependent on the wall thickness of the inner tube 12 as well as the prescribed use of the working channel 10. Typically, the ratio of outer tube 16 thickness to inner tube 12 thickness is in the range of 2:1 to 8:1. For example, if the inner tube 12 has a wall thickness of 0.004 inches, then the wall thickness of the outer tube 16 ranges between 0.008 inches and 0.032 inches. These are not critical dimensions, since the thickness of the outer tube 16 may be varied, but obviously must be within the concept of the design of its intended use or implementation to maintain a low-profile working channel 10 with high flexibility and high resistance to kinking. The ranges given are practical ranges for most applications. In the preferred embodiment, the outer tube 16 is 0.017 inches thick, so that the preferred ratio to the inner tube 12 is about 3.1:1.

ePTFE and similar materials comprise a matrix of nodes and fibers that form a porous structure. The nodes are oriented perpendicularly to the fiber, which run longitudinally through the material. The nodes are a relatively static or solid portion of the ePTFE micro-structure, while the fibers which interconnect the nodes are collapsible, allowing the outer tube 16 to undergo longitudinal compression and elongation without dimensional changes, much like the performance of a spring. It is the ratio of fiber length to node width that allows various amounts of flexion in ePTFE material. Longer fiber lengths and smaller nodes provide material with high flexibility and low radial support. Since the length of the fibers relates to the porosity of the material, the relationship between fiber lengths and material density is inverse. For example, an outer tube 16 made of ePTFE with a 25 micron fiber length could have a volume density of 0.55 g/cc, while an outer tube 16 with a 10 micron fiber length would have a density of 1.2 g/cc or higher. The volume density range for ePTFE to function in the design of the product described and shown in FIGS. 1 and 2 can range from 0.2 to 1.9 g/cc.

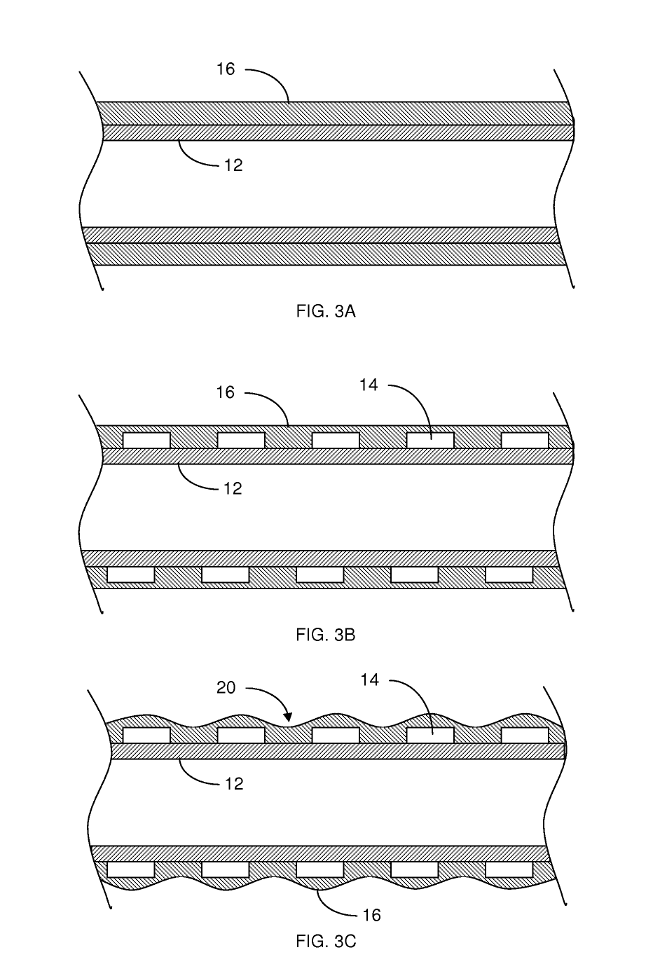

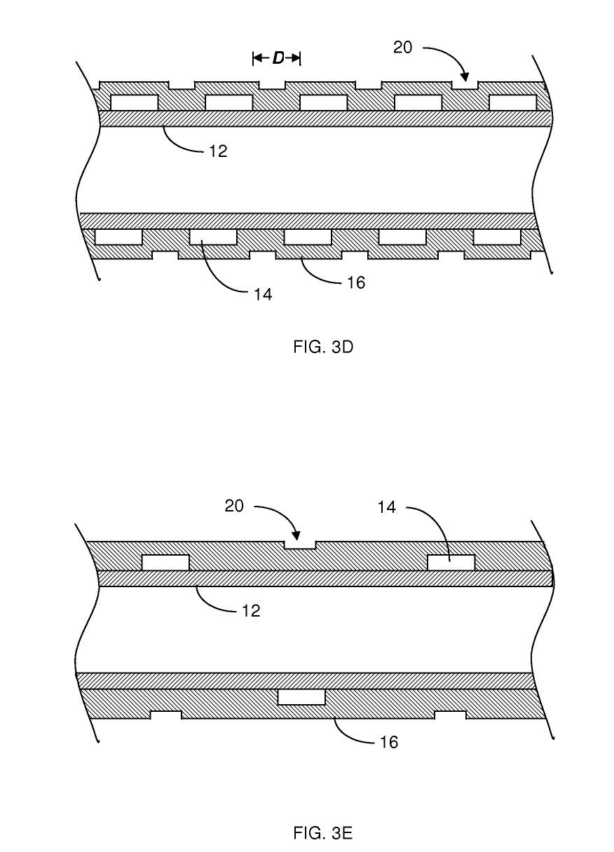

Referring to FIG. 2, at least one support wire 14 may be helically wrapped around the inner tube 12 in a single spiral wrap. The support wire 14 may be any shape in cross-section, but is preferably rectangular, having dimensions that make it substantially flat or ribbon-shaped. The support wire 14 is between 0.01 and 0.25 inches wide, most preferably between 0.01 and 0.03 inches wide, and between 0.001 and 0.004 inches thick. The support wire 14 is preferably made of stainless steel. Preferably, the support wire 14 is wrapped around the inner tube 12 with a substantially uniform spacing, illustrated by dimension D in FIG. 3D. The desired spacing width depends on the embodiment of the working channel 10, and is typically in the range of 0.006 inches to about ten times the width of the support wire 14 depending on the design requirements. The characteristics of the working channel 10 that may impact the spacing of the support wire 14 include the inner tube 12 thickness, the total wall thickness of the completed working channel 10, the support wire 14 diameter and width, and the density and thickness of the outer tube 16. The spacing is chosen in order to meet the desired flexibility and life cycle intended for the use to which the finished working channel 10 is to be placed. The support wire 14 functions as a spring and also provides radial support to the working channel 10 during flexion. The support wire 14 also provides compression resistance and radial support during subsequent manipulation and internal pressurization of the working channel 10 when it is placed in use. Thus, the support wire provides resistance to kinking and ovalling.

The inner tube 12 and outer tube 16 are bound together, encapsulating the support wire 14 if one is used. The binding may be effected during extrusion or initial sintering of the tubes 12, 16, but preferably the tubes 12, 16 are bound using a second sintering process performed after the tubes 12, 16 are formed. After the support wire 14 is wrapped around the inner tube 12 and the outer tube 16 is placed over the support wire 14 and inner tube 12, a compression wrap 18 is spirally wrapped around the outer tube 16. The compression wrap 18 covers as much as 100% of the external surface area of the outer tube 16, depending on the intended design, described below with reference to FIGS. 3A-E. After the compression wrap 18 has been spirally wound in place, the assembly, including the compression wrap 18, is heated to a temperature at or above the sintering point of both the inner tube 12 and outer tube 16. The typical sintering point for the described materials is about 320 degrees Celsius. The compression wrap 18 brings the outer tube 16 into contact with the support wire 14 and the inner tube 12. Before the materials reach the sintering point, and preferably when the materials reach about 300 degrees Celsius, one or both of the tubes 12, 16 become sufficiently pliable that heat bonding of the tubes 12 and 16 takes place where they contact one another between adjacent turns of the support wire 14. At the same time, the support wire 14 is firmly encapsulated or sandwiched between the two tubes 12, 16 as a result of the bonding which takes place. The assembly is then allowed to cool. Once cool, the compression wrap 18 is removed and discarded or reused on subsequent assemblies.

In the preferred embodiment, the compression wrap 18 is a metal, preferably stainless steel, wire about 0.025 inches wide and about 0.003 inches thick, and fully covers the external surface of the outer tube 16 by being wrapped around the outer tube 16 with approximately zero spacing. See FIGS. 1 and 3A-B. Zero spacing may be achieved by ensuring each turn of the compression wrap 18 abuts the previous turn, or by slightly or substantially overlapping the turns. The preferred result is therefore a substantially smooth external surface on the outer tube 16. Depending on the compression wrap 18 used and potentially varying factors, such as nonuniform heating, in the bonding process, the external surface of the outer tube 16 may have slight corrugations or be otherwise unsmooth. See FIG. 3C. The working channel 10 may be fed through a compression die, such as a heat sizing die, to further refine the external surface of the outer tube 16 until it is substantially or completely smooth.

In alternative embodiments, the compression wrap 18 may be a heat-conducting metal wire, such as stainless steel, having a round to substantially flat cross-section. Referring to FIGS. 2 and 3C-E, where the compression wrap 18 covers less than the full external surface area of the outer tube 16, a spiral-shaped depression 20 is formed in the region that the compression wrap 18 does cover. The depression 20 creates a corrugated external surface that increases the flexibility of the working channel 10 while still aiding in the boding process between the tubes 12, 16. In these illustrated embodiments and other embodiments where such a corrugated exterior is desired, the compression wrap 18 is a round or substantially flat wire wrapped with substantially uniform spacing around the outer tube 16. When a support wire 14 is used, the spacing of the compression wrap 18 may be the same as the spacing between adjacent turns of the support wire 14, but offset so that the compression wrap 18 is positioned in the space between adjacent turns of the support wire 14.

While the depression 20 increases flexibility, it may cause complications that a smooth external surface could avoid. The depression 20 therefore has a depth and shape that is dependent on the intended use of the working channel 10. A deep depression 20, illustrated in FIG. 3C, typically has a depth of approximately 0.001 inch. A shallow depression 20 like those shown in FIGS. 3D-E may be about 0.0005 inches deep and strikes a balance between increased flexibility and smooth exterior. Further, a compression wrap 18 comprising a round wire will form a u-shaped depression 20 such as the depression 20 shown in FIG. 3C, while a flat-wire compression wrap 18 will form a depression with a flat bottom and vertical or slightly curved sides. See FIGS. 3D-E. Generally, a u-shaped depression 20 will provide a higher degree of flexibility than a flat-bottomed depression 20.

The finished working channel 10 has the opposing ends of the tubes 12 and 16 cut co-planar to a plane which is perpendicular to the common central axis of the tubes 12, 16. One or both of the opposing ends of the finished product may be chemically etched using an etcher suitable for use with PTFE, such as that sold under the trademarks FLUOROETCH by Acton Technologies or TETRAETCH by W.L. Gore Associates. Chemical etching facilitates subsequent adhesive bonding of the etched end with the tip of an endoscope. The end of the working channel 10, which is intended to be the distal end, may be chemically etched in order to increase the capacity of the tubes 12, 16 to accept an adhesive bond at the distal section of an endoscope. The proximal end of the working channel 10 is not etched, as it typically is mechanically coupled to a proximal section of an endoscope. Alternatively, both ends of the working channel 10 may be chemically etched to increase their capacity to be adhesively bonded to a pre-existing working channel of the endoscope.

The completed working channel 10 has highly lubricious internal and external surfaces capable of a tight bend radius and a relatively low wall profile. The support wire 14 provides added resistance to kinking. In addition, the completed structure is chemical resistant and is resistant to wear or collapse during repeated flexion. The overall thickness of the finished structure typically is between 0.014 inches and 0.058 inches. The lubriciousness of each surface is determined by the coefficient of friction of the material used in the tubes 12, 16. PTFE and ePTFE are chemically resistant to most acids, bases, alcohols and so forth, and have temperature resistances of up to about 300 degrees Celsius. As described, the outer wall of the inner tube 12 and the inner wall of the outer tube 16 are bonded together via temperature and pressure, and require no adhesives or chemicals to create the bond between the tubes 12, 16. However, an adhesive may be used in place of the heat and pressure boding process if the intended use of the working channel 10 provides acceptable choices for such an adhesive. An example of an acceptable adhesive for use on a medical device is Loctite® 401 Prism used in combination with Loctite® 770 primer.

It has been found that completed units constructed according to the embodiment of FIG. 2 have an average cycle life of 5,000 cycles along the minimum bend radius of the completed tubing. In addition, completed units have been found to be capable of up to 80 PSI interior air pressures without more than 5% radial diameter deflection or leaking. Once again, the encapsulated support wire 14 improves this stability over structures which do not include the support wire 14. The support wire 14 is optional because it increases the costs associated with manufacturing and installing the working channel 10. Within the range of the described structures, it also has been found that there are no more than three percent radial deflection at a one-half inch radius. This has been found to approximate a maximum bend condition which may occur during use of the device.

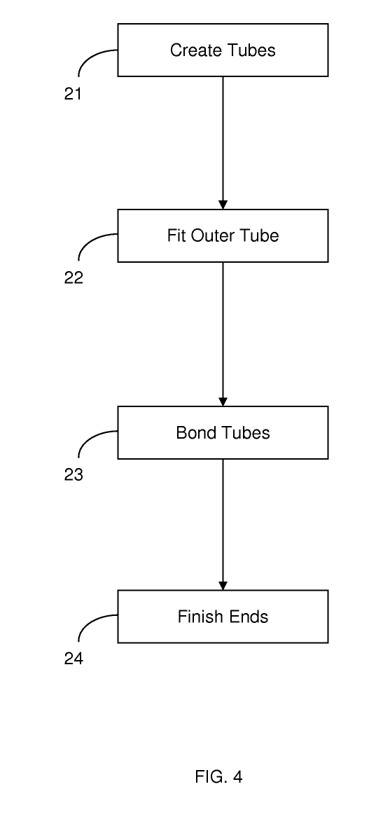

FIGS. 4-6 illustrate methods for manufacturing a working channel 10. The winding of the support wire 14 is illustrated as optional because a working channel 10 that does not have a support wire 14 can be manufactured using these methods by simply omitting the step of winding the support wire 14 onto the inner tube 12. Referring to FIG. 4, the inner and outer tubes 12, 16 are created 21 by shaping the tubes 12, 16 through extrusion or other means. Once shaped, one or both tubes 12, 16 may be solidified by sintering. The tubes 12, 16 are substantially the same length. The inner tube 12 is made with its final desired properties, such as density and wall thickness, while the outer tube 16 is made so that it will expand to assume its final desired properties. The expansion ratio of the outer tube 16 is determined by several factors, but primarily by the density given to the outer tube 16 during extrusion. The density may be further altered after creation 21 by expanding the extruded ePTFE before the material is sintered, or by repeatedly feeding the ePTFE through a compression die, before or after sintering, until a certain density is reached. In order to expand the outer tube 16, the extruded ePTFE is subjected to expanding temperatures of between 150 and 300 degrees Celsius, with longitudinal tension applied to the ends of the outer tube 16 during heating to increase the length of the outer tube 16.

The outer tube 16 is then fit 22 over the inner tube 12. As described below, the outer tube 16 may be fit 22 over the inner tube 12 after the inner tube 12 is placed on a mandrel, but use of a mandrel is optional. The inner and outer tubes 12, 16 are then bonded 23 together. Once bonding 23 is complete, the ends of the working channel 10 are finished 24, such as by chemical etching.

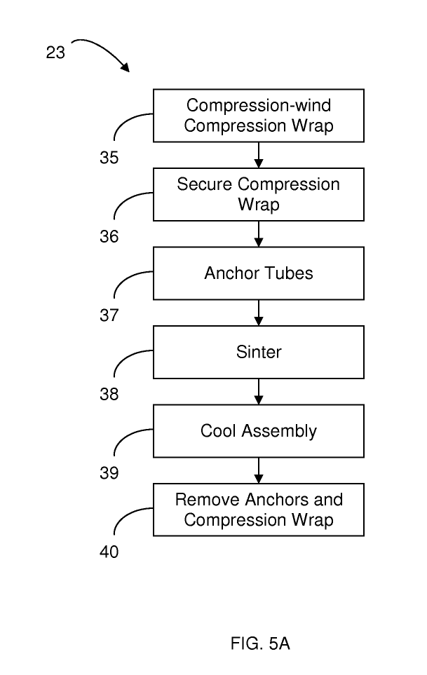

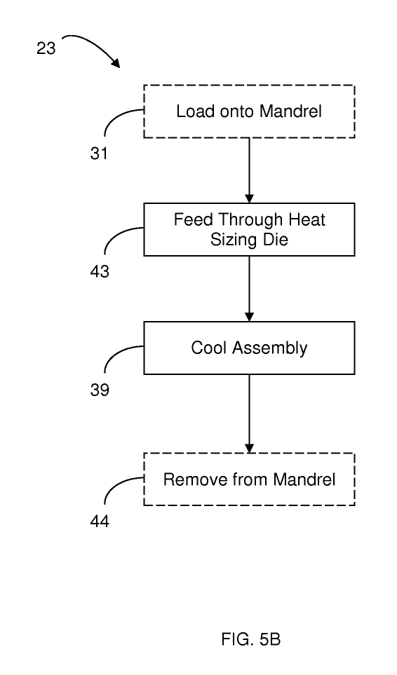

FIGS. 5A-B illustrate different methods of bonding 23 the inner and outer tubes 12, 16 together. Referring to FIG. 5A, a compression winding 35 of the compression wrap 18 is performed with sufficient pressure so as to compress the portions of the outer tube 16 beneath the compression wrap 18 as the winding takes place. This in turn supplies pressure between the inner diameter of the outer tube 16 and the outer diameter of the inner tube 12. This also produces some compression of the pores of the outer tube 16 beneath each of the turns of the compression wrap 18. Once the compression wrap 18 is wound 35, its ends are secured 36 to the mandrel by means of a removable tape or any suitable material that will hold it in place during final sintering. The tubes 12, 16 may be anchored 37 to the mandrel, such as by slipping brass rings or other fasteners over either end, to prevent longitudinal retraction of the tubes 12, 16 during sintering. The entire assembly then undergoes final sintering 38 in a heat sintering oven, which may be in the form of a convection air oven, induction heater, or furnace at a processing temperature sufficiently high to meet or exceed sintering point of the material used for the tubes 12, 16. The time duration for this sintering 38 process is approximately one to two minutes duration per foot of the assembly. This time, however, may be varied in accordance with the particular parameters of the oven used and the manner in which heat is applied to the assembly during the sintering 38 process. The assembly is then allowed to cool 39 and the anchors and compression wrap 18 are removed 40.

Referring to FIG. 5B, the inner and outer tubes 12, 16 may be bonded by optionally loading 31 the tubes 12, 16 onto a mandrel. The loading 31 may also be done before the outer tube 16 is fit 22 over the inner tube 12, or no mandrel may be used. The tubes 12, 16 are then fed 43 through a compression die, such as a heat sizing die. The compression is sufficient to cause the outer tube 16 to bond to the inner tube 12. The addition of heat hastens the bonding process and may form a stronger bond by causing the outer tube 16 to expand against the die and the inner tube 12. The assembly is allowed to cool 39 before it is removed 44 from the mandrel if one is used. This process may be used on its own to bond 23 the tubes 12, 16 as described, and may also be used subsequently to the compression-wrapping 35 method to further smooth the external surface of the outer tube 16 if desired.

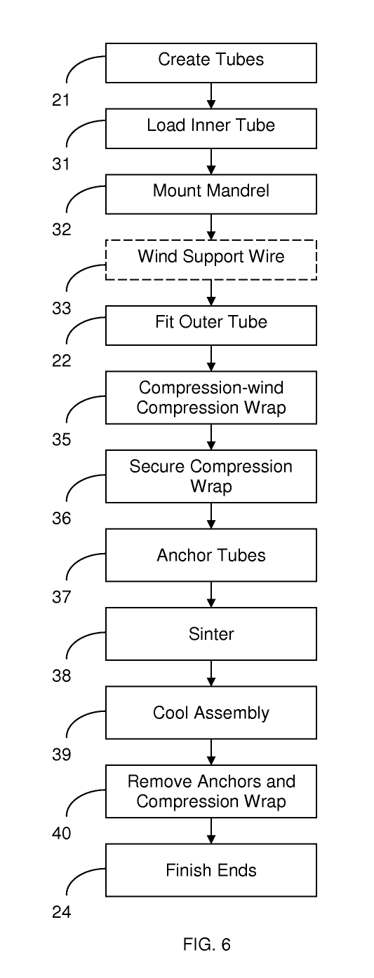

FIG. 6 illustrates the preferred method of manufacturing the working channel 10. After the tubes 12, 16 are created 21, the inner tube 12 is loaded 31 onto a mandrel, which is a rod of stainless steel, brass or aluminum, having a length which preferably is greater than the length of the finished working channel 10. The mandrel is then mounted 32 for rotation in a conventional spiral winding machine. The support wire 14 is then tightly wound 33 in a helical pattern onto the inner tube 12, from one end to the other. The winding 33 of the support wire 14 is done with sufficient pressure to firmly grip the external surface of the inner tube 12.

After the support wire 14 has been wound 33 on the inner tube 12, the outer tube 16 is fit 22 over the inner tube 12 and the support wire 14. It should be noted that the inner diameter of the outer tube 16 is equal to or slightly greater than the outer diameter of the inner tube 12 covered by the spiral winding of the support wire 14. The spiral winding machine is used to perform a compression winding 35 of the compression wrap 18 as described above. Non-compressed spaces may be left between adjacent turns of the compression wrap 18 if corrugation of the outer tube 16 is desired. The ends of the compression wrap 18 are secured 36 and the tubes 12, 16 are anchored 37 to the mandrel. Final sintering 38 is performed, and the assembly is then allowed to cool 39. Then, the anchors and compression wrap 18 are removed 40 and the two ends of the assembly are finished 24.

An alternative method of making a working channel 10 with no depression 20 in the external surface is to apply the above-described sintering process without compression-winding 35 the compression wrap 18 onto the outer tube 16 prior to final sintering 38. In this embodiment, steps 35 and 36 and the removal of the compression wrap 18 in the process described above and illustrated in FIG. 3 would be eliminated.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

I claim:

1. A working channel comprising:
a. a nonporous inner tube having a discrete length; and
b. a porous outer tube having substantially the same length as the inner tube, the outer tube being fit over the inner tube and bonded to the inner tube.

2. The working channel of claim 1 wherein the outer tube is made of expanded polytetrafluoroethylene.

3. The working channel of claim 2 wherein the outer tube has a substantially smooth external surface.

4. The working channel of claim 3 wherein the substantially smooth external surface of the outer tube is formed by winding a compression wrap around the outer tube before the outer tube is bonded to the inner tube, the compression wrap being wound so that there is substantially no space between adjacent turns of the compression wrap.

5. The working channel of claim 3 wherein the smooth external surface of the outer tube is formed by feeding the inner tube and outer tube through a compression die.

6. The working channel of claim 2 wherein the outer tube has a corrugated external surface.

7. The working channel of claim 6 wherein the corrugated external surface of the outer tube is formed by winding a compression wrap around the outer tube before the outer tube is bonded to the inner tube, the compression wrap being wound so that there is space between adjacent turns of the compression wrap.

8. The working channel of claim 1 further comprising a support wire wound helically around the inner tube, the outer tube being fit over both the support wire and the inner tube and bonded to the inner tube between adjacent turns of the support wire.

9. The working channel of claim 8 wherein the outer tube has a substantially smooth external surface.

10. The working channel of claim 9 wherein the substantially smooth external surface of the outer tube is formed by winding a compression wrap around the outer tube before the outer tube is bonded to the inner tube, the compression wrap being wound so that there is no space between adjacent turns of the compression wrap.

11. A method of making a working channel, the method comprising:
a. fitting a porous outer tube over a nonporous inner tube, the outer tube and inner tube having substantially the same length; and
b. bonding the outer tube to the inner tube.

12. The method of claim 11 wherein bonding the outer tube to the inner tube comprises feeding the inner tube and outer tube through a compression die.

13. The method of claim 12 wherein the compression die applies sufficient pressure to bond the outer tube to the inner tube.

14. The method of claim 12 wherein bonding the outer tube to the inner tube further comprises heating the outer tube and inner tube until the outer tube heat bonds to the inner tube.

15. The method of claim 12 wherein the compression die makes the external surface of the outer tube substantially smooth.

16. The method of claim 11 wherein bonding the outer tube to the inner tube comprises:
a. winding a compression wrap onto the external surface of the outer tube;
b. heating the outer tube and inner tube until the outer tube heat bonds to the inner tube;
c. allowing the outer tube and inner tube to cool; and
d. removing the compression wrap.

17. The method of claim 16 wherein the compression wrap is wound leaving no space between adjacent turns of the compression wrap, so that the external surface of the outer tube is substantially smooth after bonding.

18. The method of claim 16 wherein the compression wrap is wound leaving space between adjacent turns of the compression wrap, so that the external surface of the outer tube is corrugated after bonding.

19. The method of claim 11 further comprising loading the inner tube onto a mandrel prior to fitting the outer tube over the inner tube, the mandrel being slightly longer than the inner tube.

20. The method of claim 19 wherein bonding the outer tube to the inner tube comprises:
a. winding a compression wrap onto the external surface of the outer tube;
b. heating the outer tube and inner tube until the outer tube heat bonds to the inner tube;
c. allowing the outer tube and inner tube to cool; and
d. removing the compression wrap.

21. The method of claim 20 wherein the compression wrap is wound leaving no space between adjacent turns of the compression wrap, so that the external surface of the outer tube is substantially smooth after bonding.

22. The method of claim 20 wherein the compression wrap is wound leaving space between adjacent turns of the compression wrap, so that the external surface of the outer tube is corrugated after bonding.

23. The method of claim 19 further comprising winding a support wire around the inner tube before fitting the outer tube over the inner tube.

24. The method of claim 23 wherein bonding the outer tube to the inner tube comprises:
a. winding a compression wrap onto the external surface of the outer tube so that the compression wrap is positioned laterally between adjacent turns of the support wire;
b. heating the outer tube and inner tube until the outer tube heat bonds to the inner tube;
c. allowing the outer tube and inner tube to cool; and
d. removing the compression wrap.

25. The method of claim 24 wherein the compression wrap is wound leaving no space between adjacent turns of the compression wrap, so that the external surface of the outer tube is substantially smooth after bonding.
